(19) United States
(12) Patent Application Publication
(10) Pub. No.: US 2012/0309056 A1
(43) Pub. Date: Dec. 6, 2012

(54) FED-BATCH PROCESS USING CONCENTRATED CELL CULTURE MEDIUM FOR THE EFFICIENT PRODUCTION OF BIOLOGICS IN EB66 CELLS

(76) Inventors: Arnaud Léon, Nantes (FR); Patrick Champion-Arnaud, Nantes (FR); Nicola Beltraminelli, Lyon (FR); Sylvana Bouletreau, Nantes (FR)

(21) Appl. No.: 13/576,971

(22) PCT Filed: Feb. 4, 2011
(86) PCT No.: PCT/EP2011/051668

§ 371 Date: Aug. 3, 2012

(30) Foreign Application Priority Data
Feb. 4, 2010 (EP) 10152700.0

(51) Int. Cl. C12N 5/071; C12P 21/08; C12N 7/00; C12P 21/00

(52) U.S. Cl. 435/69.6; 435/404; 435/349; 435/69.1; 435/235.1

(57) ABSTRACT

The present invention relates to a glucose fed-batch process using concentrated cell culture for the efficient production of biologics, such as viral vaccines and recombinant proteins. In particular, the invention relates to culturing duck embryonic derived stem cells EB66 to obtain high yield of biological products from such cells.

The present invention relates to biotechnology sciences, more specifically to areas of cell culture for the production of biologics, such as viral vaccines and recombinant proteins. In particular, the invention relates to culturing animal cells, more specifically duck embryonic derived stem cells, to obtain high levels of biological products from such cells.

The past twenty years witnessed the transformation of animal cell culture from exploratory biological products production methods to mature manufacturing processes. This is essentially true for the industrial manufacturing of recombinant proteins such as monoclonal antibodies, where cell lines like CHO (Chinese Hamster Ovary) and NS0 are now widely used for the mass production of monoclonal antibodies. On the vaccine side, the actual trend is to switch from the old egg-based production process to cell-culture platforms for the production of viral vaccines. Animal cell lines constitute a highly promising alternative to the eggs and chicken embryo fibroblasts (CEFs) production systems because it overcomes viral vaccines production bottlenecks and time constrains (e.g risks of eggs shortage due to avian flu; egg shipment plan) and has additional advantages in connection with the safety of the vaccine (no antibiotic additives present in the vaccine formulation; no toxic preservatives (such as thiomersal) needed; reduced endotoxin levels, no egg allergy issue; no risk of adventitious agent/BSE by cell culture in protein and serum free media; higher purity of virus vaccine preparation). Examples of cell lines for the production of viral vaccines are MDCK (cells derived from the kidney of Madin-Darby dog), PerC6 (cells derived from human embryonic retinal cells genetically modified by inserting the E1 genes from the human adenovirus type 5) developed by Crucell (Netherland)), VERO (cells derived from epithelial cells of kidney from African green monkey (Cercopithecus aethiops) isolate at the Chiba University in Chiba, Japan), BHK21 (Cells immortalized from baby hamster kidney cells) and duck cell lines (transgenic or not), preferably duck embryonic stem cells, such as EB66 developed by Vivalis (France).

Because of biological products complexity, the costs of production are very high and there is a constant pressure on driving production cost of biological products down. A direct mean to reduce production cost is to improve biologics production yield. The production of virus or recombinant proteins in animal cell culture is dependent upon a variety of factors. For example, the production of virus by mammalian or avian cell culture can be improved using a variety of techniques, such as media optimization, synchronization of the producer cells, increasing infectivity of producer cells, virus adaptation to producer cells etc . . . However, it remains that the basic parameters that will ultimately impact the cell line productivity are the specific productivity of said cell line, the peak viable cell concentration that is attainable with said cell line and the length of the production process.

If one can achieve, a high concentration of viable producer cells, with a maximal specific productivity, for long period of time, in a given volume, then the overall efficiency of the process would be improved to make the process economical. This is the goal of the present invention to provide a process for culturing high concentration of animal cells for preparing high-titer viral suspension and/or recombinant proteins in cell cultures.

The inventors have now find that duck cell lines, and more specifically duck EB66 cell line, have the remarkable characteristic to grow in animal serum free cell culture medium containing high concentration of sugar source (i.e glucose) up to very high cell density (>60 millions cells/ml) in fed-batch culture, without accumulation of toxic compounds in the cultum (such as lactate and ammonium), allowing to improve the overall efficiency of the process of biological pro ducts production.

The present invention relates to a process for the fed-batch culture of animal cells comprising the steps of:

a) growing the cells, preferably in exponential phase, in a cell growth medium, optionally supplemented with defined supplements, in a culture volume corresponding to a maximum of 75%, preferably to about 50% or less, of the maximum vessel volume, to a cell density greater or equal to 4 million cells/ml, more preferably to a cell density greater or equal to 6, 8, 10, 12, 15, 20, 25, 30, 35, or even 40 million cells/ml;

and wherein said process comprises the steps of:

b) performing a fed-batch culture by feeding the culture with a concentrated cell culture medium and/or at least one source of carbohydrate, preferably a sugar and/or glutamine, until the culture volume reaches the maximum vessel volume; and then

c) optionally, adding for the remaining days of animal cell culture in said vessel, marginal volume of at least one compound selected among one source of carbohydrate (preferably a sugar as an energy source) and a solution of one or more amino-acids, preferably glutamine.

According to a preferred embodiment, the present invention relates to a process for the fed-batch culture of animal cells, preferably avian cell lines, more preferably duck cell lines, and even more preferably duck EB66 cell line, comprising the steps of:

a)—growing the cells, preferably in exponential phase, in a cell growth medium, optionally supplemented with defined supplements, in a culture volume corresponding to about 50% or less of the maximum vessel volume, to a cell density greater or equal to 10 million cells/ml, preferably 12 million cells/ml, more preferably 15 million cells/ml, or even more preferably to a cell density greater or equal to 20 million cells/ml;

and wherein said process comprises the steps of:

b)—performing a fed-batch culture by feeding the culture with a concentrated cell culture medium, D-glucose and glutamine, until the culture volume reaches the maximum vessel volume; and then

c) optionally, adding for the remaining days of animal cell culture in said vessel, marginal volume of D-glucose and glutamine.

According to a preferred embodiment, the cell density at the end of step b) is greater or equal to 10 million cells/ml, preferably 15 million cells/ml, more preferably 20 million cells/ml, or even more preferably greater or equal to 25 million cells/ml.

In the process of the invention, step a) is a batch cell culture, steps b) and c) is a fed-batch cell culture. In a second embodiment, the process of the invention, steps a), b) and c) are a fed-batch cell culture. As used herein, the term “batch” describes a batch cell culture which is carried out by placing the cells to be cultured in a fixed volume of culture medium and allowing the cells to grow. Cell numbers increase, usually exponentially, until a maximum is reached, after which growth become arrested and the cells die. This may be due either to exhaustion of a nutrient or accumulation of an inhibitor of growth. Thus, batch culture is characterised in that it proceeds in a fixed volume since nothing is added after placing the cells in the cell growth medium, optionally supplemented with defined supplements. As used herein, the term “fed-batch” describes a batch cell culture (i.e cells are cultured in a medium in a fixed volume) to which substrate, in either solid or concentrated liquid form, is added either periodically or continuously during the run. The volume of the feed is minimal compared to the volume of the culture. When the feeding is discontinuous, the feeding may occur one time per day, more than one time per day, or less than one time per day. According to a preferred embodiment, the glucose and glutamine fed-batch is continuous and the fed-batch of concentrated cell culture medium is discontinuous. According to a second preferred embodiment, the glucose and glutamine and the fed-batch of concentrated cell culture medium is continuous. Just as in a batch culture, a fed-batch culture is initiated by inoculating cells to the medium, but, in contrast to a batch culture, there is a subsequent inflow of nutrients, such as by way of a concentrated nutrient feed. In contrast to a continuous culture there is no systematic removal of culture fluid or cells from a fed-batch culture. Batch and fed-batch culture are characterized in that it proceeds in a substantially fixed volume, for a fixed duration, and with a single harvest either when the cells have died or at an earlier, predetermined point.

By “cell growth medium”, “cell culture medium” or “culture media” or “media formulation” it is meant a nutritive solution for culturing or growing cells. The ingredients that compose such media may vary depending on the type of cell to be cultured. In addition to nutrient composition, osmolarity and pH are considered important parameters of culture media.

The cell growth medium comprises a number of ingredients well known by the man skilled in the art, including amino acids, vitamins, organic and inorganic salts, sources of carbohydrate, lipids, trace elements (CuSO4, FeSO4, Fe(NO3)3, ZnSO4 . . . ), each ingredient being present in an amount which supports the cultivation of a cell in vitro (i.e survival and growth of cells). Ingredients may also include different auxiliary substances, such as buffer substances (like sodium bicarbonate, Hepes, Tris . . . ), oxidation stabilizers, stabilizers to counteract mechanical stress, protease inhibitors, animal growth factors, plant hydrolyzates, anti-clumping agents, anti-foaming agents. If required, a non-ionic surfactant, such as polypropylene glycol can be added to the cell growth medium as an anti-foaming agent. These agents are generally used to protect cells from the negative effects of aeration since, without an addition of a surfactant, the ascending and bursting air bubbles can lead to damage of those cells that are located on the surface of these air bubbles (“sparging”).

Characteristics and compositions of the cell growth media vary depending on the particular cellular requirements. Important parameters include osmolarity, pH, and nutrient formulations.

The cell growth medium is preferably an animal serum-free mediums” (SFM), which meant that the cell growth medium is ready to use, that is to say that it does not required serum addition allowing cells survival and cell growth. The cell growth medium is preferably chemically defined, but it may also contained hydrolyzates of various origin, from plant for instance. Preferably, said cell growth medium is “non animal origin” qualified, that is to say that it does not contain components of animal or human origin (FAO status: “free of animal origin”). In SFM, the native serum proteins are replaced by recombinant proteins. Alternatively SFM medium according to the invention does not contain protein (PF medium: “protein free medium”) and/or are chemically defined (CDM medium: “chemically defined medium”). SFM media present several advantages: (i) the first of all being the regulatory compliance of such media (indeed there is no risk of contamination by adventitious agents such as BSE, viruses); (ii) the optimization of the purification process; (iii) the better reproducibility in the process because of the better defined medium. Example of commercially available cell growth media are: VP SFM (InVitrogen Ref 11681-020, catalogue 2003), Opti Pro (InVitrogen Ref 12309-019, catalogue 2003), Episerf (InVitrogen Ref 10732-022, catalogue 2003), Pro 293 S-CDM (Cambrex ref 12765Q, catalogue 2003), LC17 (Cambrex Ref BESP302Q), Pro CHO 5-CDM (Cambrex ref 12-766Q, catalogue 2003), HyQ SFM4CHO (Hyclone Ref SH30515-02), HyQ SFM4CHO-Utility (Hyclone Ref SH30516.02), HyQ PF293 (Hyclone ref SH30356.02), HyQ PF Vero (Hyclone Ref SH30352.02), Ex cell 293 medium (SAFC Biosciences ref 14570-1000M), Ex cell 325 PF CHO Protein free medium (SAFC Biosciences ref 14335-1000M), Ex cell VPRO medium (SAFC Biosciences ref 14560-1000M), Excell EBx Gro-I medium (SAFC Biosciences—ref.14530c).

The cell growth medium may be supplemented with defined supplements such as antibiotic to prevent bacterial contamination. Example of antibiotics include gentamycin, penicillin and streptomycin. Gentamycin is usually used at a final concentration of 10 ng/ml, penicillin at a final concentration of 100 U/ml and streptomycin at a final concentration of 100 μg/ml. The cell growth medium may also be supplemented with defined supplements such as glutamine. Glutamine being unstable in culture medium it is often necessary to supplement the cell growth medium to adjust the glutamine concentration in the medium. Alternatively, the cell growth medium may be supplemented during the exponential phase with highly concentrated solutions of ingredients, some of them being marketed as “CHO-Feed” (SAFC cat. No C1615). According to a preferred embodiment, the cell growth medium is supplemented one time with defined supplements. According to another embodiment, the cell growth medium is supplemented periodically with defined supplements.

By concentrated cell culture medium it is meant a formulation of a cell culture medium used to grow cells, and obtained by increasing the concentration of (almost) each component of the cell culture medium. According to a preferred embodiment, the concentrated cell culture medium formulation is obtained by increasing the concentration of each component of the medium except the components which are involved in, or act on, the osmotic pressure and/or osmolarity and/or osmolality, such as salts and buffering agents. A man skilled in the art is able to define such components.

A “1× formulation” is meant to refer to any aqueous solution that contains some or all ingredients found in a cell culture media. The “1× formulation” can refer to, for example, the cell culture media or to any subgroup of ingredients for that media. The concentration of an ingredient in a 1× solution is about the same as the concentration of that ingredient found in the cell culture formulation used for maintaining or growing cells. Cell culture media used to grow cells is a 1× formulation by definition. When a number of ingredients are present (as in a subgroup of compatible ingredients), each ingredient in a 1× formulation has a concentration about equal to the concentration of those ingredients in a cell culture media. For example, RPMI 1640 culture media contains, among other ingredients, 0.2 g/l L-arginine, 0.05 g/l L-asparagine, and 0.02 g/l L-aspartic acid. A “1× formulation” of these amino acids, which are compatible ingredients, contains about the same concentrations of these ingredients in solution. Thus, when referring to a “1× formulation,” it is intended that each ingredient in solution has the same or about the same concentration as that found in the cell culture media being described. The concentrations of media ingredients in a 1× formulation are well known to those of ordinary skill in the art, See Methods For Preparation of Media, Supplements and Substrate For Serum-Free Animal Cell Culture Alan R. Liss, New York (1984). The osmolarity and/or pH, however, may differ in a 1× formulation compared to the culture media, particularly when fewer ingredients are contained by the 1× formulation.

A “10× formulation” refers to a solution wherein each ingredient in that solution is about 10 times more concentrated than the same ingredient in the cell culture media. RPMI 1640 media, for example, contains, among other things, 0.3 g/l L-glutamine. By definition, a “10× formulation” contains about 3.0 g/l glutamine. A “10× formulation” may contain a number of additional ingredients at a concentration about 10 times that found in the 1× culture media.

As will be apparent, 1.5×, 2×, 3×, 4×, 5×, 6×, . . . 10×, . . . 25× formulation etc . . . designate solutions that contain ingredients at about 1.5, 2, 3, 4, 5, 6, 10 or 25 fold concentrations, respectively, as compared to a 1× cell culture media. Again, the osmolarity and pH of the media formulation and concentrated formulation may vary. The solubility of some components in cell culture medium constitutes a limitation to the obtaining of highly concentrated cell culture medium.

In a fed-batch culture, the concentrated cell culture medium is typically rather concentrated to minimize the increase in culture volume while supplying sufficient nutrients for continued cell growth. More preferably, concentrated cell culture medium of the invention is 3×, 4×, 5×, or 6×.

The concentrated cell culture medium used for the fed-batch culture may be in a concentrated cell growth medium as defined above, or may comprise at least one ingredients of the cell growth medium. According to a first embodiment the concentrated cell culture medium is a concentrated formulation of the cell growth medium except that the concentration of components which are involved in, or act on, the osmotic pressure and/or osmolarity and/or osmolality (such as salts and buffering agents) remain at 1× concentration. According to a second embodiment the concentrated cell culture medium is a concentrated formulation of cell culture medium which is different from the cell growth medium.

According to another embodiment, the concentrated cell culture medium used for the fed-batch culture may comprise at least one ingredient. Ingredients are selected from the group consisting of amino-acids, lipids, carbohydrates, protein hydrolyzates of non-animal origin, surfactant, salts, trace elements and a mixture thereof. When the ingredient is amino acids, the choice of amino-acid(s) to add to the cell culture may be determined be an analysis of amino-acids consumption by the cells in the culture. By amino acid is intended all naturally occurring alpha amino acids in both their D and L stereoisomeric forms, and their derivatives. A derivative is defined as an amino acid that has another molecule or atom attached to it. Generally the amino acids are present in the cell growth medium at the start of the culture, but some amino acids may be depleted faster that others depending of cell growth medium; it is therefore required to feed with some amino acids. The non exhaustive list of amino acids (including salts and precursors) may be added to the culture medium glutamine, asparagine, cystine, tyrosine, tryptophan, lysine, histidine, arginine, glycine, valine, methionine, threonine, serine, isoleucine, leucine and phenylalanine. According to a preferred embodiment, the amino-acids added to the medium are selected from the group consisting of asparagine and glutamine, or a mixture thereof. In a more preferred embodiment, glutamine is added, and the feeding of glutamine is performed during step b) and c) to maintain the glutamine concentration in the medium between around 0.5 mM to around 5 mM, preferably between around 1 mM to around 3 mM, and most preferably around 2.5 mM. In a preferred embodiment, the feeding of glutamine occurs on a continuous basis.

In order to improve cell growth, but also viral or recombinant proteins production, additional ingredients are preferably added to the cell growth medium as a feed (i.e fed-batch process). According to a preferred embodiment, the carbohydrates are added to the medium as a feed. The carbohydrates are selected from the group consisting of D-glucose, D-sucrose and D-galactose or a mixture thereof. According to a more preferred embodiment, the carbohydrate added is D-glucose. The feeding of D-glucose is performed preferably during steps b) and c) to maintain the D-glucose concentration in the medium between around 0.5 g/l to 25 g/l of D-glucose, preferably between around 1 g/l to 15 g/l of D-glucose, preferably above 8 g/l of D-glucose, and even most preferably above 10 g/l. In a preferred embodiment, the feeding of D-glucose occurs on a continuous basis. According to a preferred embodiment, the glucose concentration in the cell growth medium is maintained by fed-batch at a final concentration of at least 8 g/l.

According to another preferred embodiment, glutamine is added to the medium as a feed (i.e fed-batch process). Glutamine is known that glutamine is unstable in culture medium. Since the degradation of glutamine in culture medium may be cytotoxic (specially ammonia and pyrrolidine carboxylic acid), it is therefore important to adjust the feeding rate to avoid accumulation of cytotoxic compounds that may cause lower yields and productivities. The feeding of glutamine is performed preferably as previously mentioned. According to a preferred embodiment the glutamine concentration in the medium is maintained by fed-batch at a final concentration of about 2.5 mM.

According to the fed-batch process of the invention, the maximum vessel volume is reached at day 5 post vessel seeding, preferably at day 6, 7, 8, 9, 10, 11, or 12 post-vessel seeding. According to a preferred embodiment, the fed-batch process of the invention, the maximum vessel volume is reached at day 6 post-vessel seeding.

According to a preferred embodiment, the process of the invention lasts less than 15 days, more preferably 12 days post-vessel seeding. According to a preferred embodiment, step a) of the process lasts 2 to 5 days, more preferably 3 or 4 days, and even more preferably 3 days.

By “marginal volume”, it is meant that the volume of compound added to the vessel is negligible compared to the maximum volume of the vessel which is recommended by the manufacturer. For example, if the maximum volume of the vessel is 2 L, adding 1 to 10 ml of compounds into the vessel, will be considered as a marginal volume increase.

By growing the cells in exponential phase, also called the log phase, it is meant a period characterized by cell doubling. In this phase, the number of new cells appearing per unit time is proportional to the present population. If growth is not limited, doubling will continue at a constant rate so both the number of cells and the rate of population increase doubles with each consecutive time period. In this phase, the period of growth up to the point at which maximum viable cell population density is reached. For this type of exponential growth, plotting the natural logarithm of cell number against time produces a straight line. The slope of this line is the specific growth rate of the organism, which is a measure of the number of divisions per cell per unit time. The actual rate of this growth (i.e. the slope of the line) depends upon the growth conditions, which affect the frequency of cell division events and the probability of both daughter cells surviving. Exponential growth cannot continue indefinitely, however, because the medium is soon depleted of nutrients and enriched with wastes.

By “vessel” it is meant, glass, plastic or metal containers of various sizes that can provide an aseptic environment for growing cells are termed “culture vessels”. The cultivation vessel of the invention is more preferably selected among stirred tank bioreactor, disposable bioreactor (such as Wave™ Bioreactor, Bello™ bioreactor, Nucleo™ bioreactor etc . . . ), spinner flask, shaken Erlenmeyer, tissue culture flasks, Roller Bottles and a cell factory. According to a preferred embodiment, the vessel is a stirred tank stainless steel or disposal bioreactor that allows control of temperature, aeration, pH and other controlled conditions and which is equipped with (i) appropriate inlets for introducing the cells, sterile oxygen, various media for cultivation, etc.; (ii) outlets for removing cells and media; and (iii) means for agitating the culture medium in the bioreactor.

Cells cultured according to the present method are in a “suspension state.” A “suspension” of cells is to be broadly understood as including all types of suspended or dispersed cell cultures; the term “suspension state” is thus used to distinguish cells that are not cultured in a liquid medium, such as cells cultured by way of adhering on a support (e.g Petri dish or tissue culture flasks). Thus, the term “suspension” includes both freely dispersed cells and agglomerated cells, regardless of whether agglomeration occurs spontaneously or as a result of some exogenously supplied nucleating factor or agent.

As used herein, the term “animal cells” or “animal cell line” describes eukaryotic cells, and more preferably vertebrate cells, and even more preferably mammalian cells and avian cells. In the present invention, the terms “cell line” and “cells” will be used indistinctly. Animal cell lines may be genetically modified or not.

A number of mammalian cell lines are well known in the art and include for example cell lines derived from:
human cells (e.g. PER.C6 cells which are described, for example, in WO01/38362, WO01/41814, WO02/40665, WO2004/056979, and WO2005/080556), MRC-5 (ATCC CCL-171), WI-38 (ATCC CCL-75), HEK cells, HeLa cells, fetal rhesus lung cells (ATCC CL-160), human embryonic kidney cells (293 cells); or
non-human primate (e.g. monkey) cells (e.g Vero cells derived from monkey kidneys),
dog (e.g. MDCK cells from dog kidneys (as described in WO 97/37000 and WO 97/37001);
rodent (e.g. hamster cells, such as BHK21-F, HKCC cells, or Chinese hamster ovary (CHO) cells).

As an alternative to mammalian sources, cell lines for use in the invention may be derived from avian sources such as chicken, duck, goose, quail or pheasant. The term “avian” as used herein is intended to have the same meaning as “bird”, “aves” or “ava”, and will be used indistinctly. “Avian” refers to any species, subspecies or race of organism of the taxonomic class <<ava>>. In a preferred embodiment, “avian” refers to any animal of the taxonomix order “Anseriformes” (i.e duck, goose, swan and allies), “Galliformes” (i.e chicken, quails, turkey, pheasant and allies) and “Columbiformes” (i.e Pigeon and allies).

Avian cell lines may be derived from a variety of developmental stages including embryonic, chick and adult. Avian cell lines may be genetically modified or not. Preferably, the cell lines are derived from the embryonic cells, such as embryonic stem cells, embryonic fibroblasts, germ cells, or individual organs, including neuronal, brain, retina, kidney, liver, heart, muscle, or extra-embryonic tissues and membranes protecting the embryo. Examples of avian cell lines include avian embryonic stem cells (WO01/85938 and WO03/076601), immortalized duck retina cells (WO2005/042728), genetically modified avian cells expressing telomerase reverse transcriptase (WO2007/077256 and WO2009/004016). Suitable avian embryonic derived stem cells, include the EBx cell lines derived from chicken embryonic stem cells such as EB45, EB14 and EB14-074 (WO2006/108846) or derived from duck embryonic stem cells, such as EB66, EB26, EB24 (WO2008/129058 & WO2008/142124). More preferably, the duck cell line is EB66 cell line.

Duck cell lines, specially EB66 cell line, Hybridoma cells (e.g NS0, YB2/0), CHO cells, Baby Hamster Kidney (BHK) cells, PerC6 cells and 293 cells, are particularly well-suited for use in the method of the invention.

Cells cultured according to the present method may be genetically modified or not by using recombinant DNA technology.

Production of Virus

The process of the fed-batch culture of animal cells is particularly suited for the replication and production of human and animal viruses in the cultured animal cells for the manufacture of human and animal vaccines.

The present invention relates to a process of production of virus in a fed-batch culture of animal cells comprising the steps of:

a)—growing the cells, preferably in exponential phase, in a cell growth medium, optionally supplemented with defined supplements, in a culture volume corresponding to a maximum of 75%, preferably to about 50% or less, of the maximum vessel volume, to a cell density greater or equal to 4 million cells/ml, more preferably to a cell density greater or equal to 6, 8, 10, 12, 15, 20, 25, 30, 35, or even 40 million cells/ml;

b)—performing a fed-batch culture by feeding the culture with a concentrated cell culture medium and/or at least one source of carbohydrate, preferably a sugar and/or glutamine, until the culture volume reaches the maximum vessel volume; and then

c) optionally, adding for the remaining days of animal cell culture in said vessel, marginal volume of at least one compound selected among one source of carbohydrate, preferably a sugar as an energy source and a solution of one or more amino-acids, preferably glutamine.

and wherein said process comprises the step of infecting the culture of animal cells with said virus, and wherein the virus infection step is carried out either during step a), between steps a) and b), or during step b).

According to a first preferred embodiment, the virus infection step is carried during step b). According to a second preferred embodiment, the virus infection step is carried between steps a) and b).

According to a preferred embodiment, the present invention relates to a process of production of virus in a fed-batch culture in animal cells, preferably duck cell lines, and more preferably duck EB66 cell line, comprising the steps of:

a)—growing the cells, preferably in exponential phase, in a cell growth medium, optionally supplemented with defined supplements, in a culture volume corresponding to about 50% or less of the maximum vessel volume, to a cell density greater or equal to 10 million cells/ml, preferably 12 million cells/ml, more preferably 15 million cells/ml, or even more preferably to a cell density greater or equal to 20 million cells/ml;

b)—performing a fed-batch culture by feeding the culture with a concentrated cell culture medium, D-glucose and glutamine, until the culture volume reaches the maximum vessel volume; and then

c) optionally, adding for the remaining days of animal cell culture in said vessel, marginal volume of D-glucose and glutamine.

and wherein said process comprises the step of infecting the culture of animal cells with said virus, and wherein the virus infection step is carried out either during step a), between steps a) and b), or during step b).

According to a preferred embodiment, when producing viruses, the cells of step a) are grown to a cell density greater or equal to 8 million cells/ml, greater or equal to 10 million cells/ml, more preferably greater or equal to 15 million cell/ml, and even more preferably greater or equal to 20 million cell/ml.

For some viruses, such the virus families of paramyxoviridae (i.e Newcastle disease virus) or orthomyxoviridae (i.e Influenza virus), the fed-batch process of virus production comprises the additional step of adding proteolytic enzyme in the culture medium in conditions that allow or favor virus propagation. The proteolytic enzyme is selected from the group consisting of trypsin, chymotrypsine, thermolysine, pepsine, pancreatine, papaïne, pronase, subtilisin A, elastase, furine and carboxypeptidase. According to a preferred embodiment, the enzyme is trypsin. The final concentration of trypsin in cell culture medium is comprises between around 0.5 to 1 mg/ml up to 25 mg/ml. More preferably, the final concentration of trypsin in cell culture medium is comprised between 0.01 to 10 usp/ml (usp: US pharmacopea unit) preferably around between 0.05 to 2 usp/ml, more preferably around between 0.3 to 1 usp/ml. Preferably, the proteolytic enzyme is a recombinant protein produced on a procaryotic host. According to a preferred embodiment, the proteolytic enzyme is added before, during and after the virus infection. According to a preferred embodiment, the proteolytic enzyme is added once daily during step b) until virus harvest.

The term “virus” as used herein includes not only naturally occurring viruses but also attenuated viruses, re-assortant viruses, viral vaccine strains, as well as recombinant viruses and viral vectors, and so on. The virus of the invention are preferably selected from the group consisting of adenoviruses, hepadnaviruses, herpes viruses, orthomyxoviruses, papovaviruses, paramyxoviruses, picornaviruses, poxviruses, reoviruses and retroviruses.

In a preferred embodiment, the viruses, the related viral vectors, viral particles and viral vaccines belong to the family of poxviridae, and more preferably to the chordopoxviridae. In one embodiment, the virus or the related viral vectors, viral particles and viral vaccines are a poxvirus, preferably an avipoxvirus selected among fowlpox virus (i.e TROVAC), canarypox virus (i.e ALVAC), juncopox virus, mynahpox virus, pigeonpox virus, psittacinepox virus, quailpoxvirus, sparrowpoxvirus, starling poxvirus, turkeypox virus. According to another preferred embodiment, the virus is a vaccinia virus selected among Lister-Elstree vaccinia virus strain, modified vaccinia virus such as Modified Vaccinia virus Ankara (MVA) which can be obtained from ATCC (ATCC Number VR-1508), NYVAC (Tartaglia et al., 1992, Virology, 188:217-232), LC16m8 (Sugimoto et Yamanouchi, 1994, Vaccine, 12:675-681), CVI78 (Kempe et al., 1968, Pediatrics 42:980-985) and other recombinant or non-recombinant vaccinia virus.

In another preferred embodiment, the viruses, the related viral vectors, the viral particles and vaccines belong to the family of ortho-myxoviridae, in particular influenza virus. The influenza virus is selected from the group consisting of human influenza virus, avian influenza virus, equine influenza virus, swine influenza virus, feline influenza virus. Influenza virus is preferably selected in strains A, B and C. Among strains A, one can recite viruses with different subtypes of haemagglutinin and neuraminidase, such as without limitation H1N1, H2N2, H3N2, H4N2, H4N6, H5N1, H5N2, H7N7 et H9N2. Among H1N1 strains, one can recite A/Porto Rico/8/34, A/New Caledonia/20/99, A/Beijing/262/95, A/Johannesburg/282/96, A/Texas/36/91, A/Singapore, A/Solomon Islands/03/2006. Among strains H3N2, one can recite A/Panama/2007/99, A/Moscow/10/99, A/Johannesburg/33/94, A/Wisconsin/10/04. Among B strains, one can recite without limitation B/Porto Rico/8/34, B/Johannesburg/5/99, B/Vienna/1/99, B/Ann Arbor/1/86, B/Memphis/1/93, B/Harbin/7/94, N/Shandong/7/97, B/Hong Kong/330/01, B/Yamanashi/166/98, B/Jiangsu/10/03, B/Malaysia. The influenza Virus of the invention is selected among wild type virus, primary viral isolate obtained from infected individual, recombinant virus, attenuated virus, temperature sensitive virus, low-temperature adapted virus, reassortant virus, reverse genetic engineered virus.

In another preferred embodiment, the viruses, the related viral vectors, the viral particles and vaccines belong to the family of paramyxoviridae. Preferably the virus is a naturally occurring paramyxovirus or a recombinant paramyxovirus selected in the group comprising measles virus, mumps virus, rubella virus, Sendai virus, Respiratory Syncythial virus (RSV), human para-influenza types I and III, Rinderpest virus, canine distemper virus, Newcastle disease virus, duck para-influenza virus. According to preferred embodiment, the virus is measles virus or a recombinant measles virus. According to another preferred embodiment, the virus is Newcastle Disease virus (NDV) or a recombinant NDV. Example of NDV strain is LaSota strain. When the virus of the invention is NDV, the process of the invention comprises preferably the additional step of adding proteolytic enzyme in the culture medium in conditions that allow virus propagation. According to a preferred embodiment, the enzyme is trypsin. The final concentration of trypsin in cell culture medium is comprises between around 0.01 ug/ml up to 10 ug/ml. More preferably, the final concentration of trypsin in cell culture medium is comprised between 0.01 to 10 usp/ml (usp: US pharmacopea unit) preferably around between 0.3 to 1 usp/ml, more preferably around between 0.4 to 0.75 usp/ml. Interestingly, the EBx® cell lines of the invention that may grow in adherence are useful to perform virus titration, and preferably NDV titration, on a plaque assay. Indeed, unlike CEFs and chicken DF1 fibroblasts for which is was not possible to observe any cytopathic effects, virus growth in EBx® cells leads to the formation of characteristic giant cells. In addition, NDV viral particles may be determined by haemagglutination assay. Therefore, the invention also pertain to the use of EB66 cells of the invention for the titration of virus, such as NDV virus.

In another preferred embodiment, the viruses, the related viral vectors, the viral particles and vaccines belong to the family of togaviridae. Preferably the virus is a naturally occurring alphavirus or a recombinant alphavirus selected in the group comprising Sinbis virus, Semliki forest virus, O'nyong'nyong virus, Chikungunya virus, Mayaro virus, Ross river virus, Eastern equine encephalitis virus, Western Equine encephalitis virus, Venezuelan Equine encephalitis virus.

In another preferred embodiment, the viruses, the related viral vectors, the viral particles and vaccines belong to the family of herpesviridae. Preferably the virus is a naturally occuring Marek Disease virus or a recombinant Marek Disease virus. The Marek Disease virus (MDV) is preferably selected among the license vaccine strains of MDV such as: FC126 (HTV), SB-1, 301B/1, CVI988 Clone C, CV1988/C/R6, CVI988/Rispens, R2/23 (Md11/75).

In another preferred embodiment, the viruses, the related viral vectors, the viral particles and vaccines belong to the family of hepadnaviridae. Preferably the virus is a naturally occuring naturally occuring hepadnavirus or a recombinant hepadnavirus, preferably selected among avian and human hepadnavirus. The avian hepadnavirus is preferably selected among the group consisting of duck hepatitis B virus (DHBV), heron hepatitis B virus (HHBV) and snow goose (SGHBV).

In another preferred embodiment, the viruses, the related viral vectors, the viral particles and vaccines belong to the family of birnaviridae, in particular Infectious Bursal Disease virus.

In another preferred embodiment, the viruses, the related viral vectors, the viral particles and vaccines belong to the family of flaviviridae, in particular Dengue virus, Japanese encephalitis virus and West Nile virus.

In another preferred embodiment, the viruses, the related viral vectors, the viral particles and vaccines belong to the family of coronaviridae, in particular Infectious Bronchitis virus.

In another preferred embodiment, the viruses, the related viral vectors, the viral particles and vaccines belong to the family of circoviridae, in particular Chicken Anemia virus.

In another preferred embodiment, the viruses, the related viral vectors, the viral particles and vaccines belong to the family of retroviridae. Preferably the virus is a naturally occurring retrovirus selected among reticulo-endotheliosis virus, duck infectious anemia virus, suck spleen necrosis virus, or a recombinant retrovirus thereof.

In another preferred embodiment, the viruses, the related viral vectors, the viral particles and vaccines belong to the family of parvoviridae. Preferably the virus is a naturally occurring parvovirus such as duck parvovirus or a recombinant parvovirus thereof.

In another preferred embodiment, the viruses, the related viral vectors, the viral particles and vaccines belong to the family of adenoviridae. Preferably the virus is a naturally occurring adenovirus preferably selected among fowl adenovirus, goose adenovirus, duck adenovirus and pigeon adenovirus or a recombinant adenovirus thereof. Examples of Fowl adenovirus are Fowl adenovirus 1 (CELO), Fowl adenovirus 5 (340), Fowl adenovirus 4 (KR95), Fowl adenovirus 10 (CFA20), Fowl adenovirus 2 (P7-A), Fowl adenovirus 3 (75), Fowl adenovirus 9 (A2-A), Fowl adenovirus 11 (380), Fowl adenovirus 6 (CR119), Fowl adenovirus 7 (YR36), Fowl adenovirus 8a (TR59) Fowl adenovirus 8b (764) and Egg Drop Syndrome virus. Examples of Goose adenovirus are Goose adenovirus 1, Goose adenovirus 2, Goose adenovirus 3. Example of Duck adenovirus is Duck adenovirus 2. Example of Pigeon adenovirus is Pigeon adenovirus 1.

Recombinant viruses include but are not limited to viral vectors comprising a heterologous gene. In some embodiments, a helper function(s) for replication of the viruses is provided by the host cell, a helper virus, or a helper plasmid. Representative vectors include but are not limited to those that will infect avian or mammalian cells.

Virus production process according to the present method can, optionally, include a harvest step and/or purification steps of the produced virus. Viruses may be intracellular or extracellular. Methods of isolating and purifying these viral products include, but are not limited to, centrifugation, ultra-filtration and chromatography.

In preferred embodiment, the process of production of virus in a fed-batch culture in animal cells, is harvested at a time between 8 and 20 days (from the vessel seeding). It is expected that after 8 days already some increase in yield will be obtained as compared to the batch process, whereas processes longer than 20 days would likely suffer from a decrease in quality of the product obtained. The duration of the fed-batch process of virus production will depend of the virus.

The production of virus in a fed-batch culture in animal cells according to the invention is performed at a temperature comprises between 32° C. to 39° C. depending of the virus type. According to a preferred embodiment, step a) is performed at a temperature comprise between 35 and 39° C., more preferably approximately 37° C.; step b) and c) are performed at a lower temperature comprises below 37° C., preferably below 35° C., more preferably below 34° C., more preferably below 33° C., and even more preferably below or at 32° C.

The invention also relate to the virus produce by the process of the invention. The instant invention also relates to the vaccine containing the virus of the invention.

Production of Recombinant Proteins

The process of the fed-batch culture of animal cells is particularly suited for the production of recombinant protein in the cultured animal cells for the manufacture of human and animal therapeutic and prophylactic drugs.

The present invention relates to a process of production of recombinant protein, such as monoclonal antibody, in a fed-batch culture of animal cells comprising the steps of:

a)—growing the cells, preferably in exponential phase, in a cell growth medium, optionally supplemented with defined supplements, in a culture volume corresponding to a maximum of 75%, preferably to about 50% or less, of the maximum vessel volume, to a cell density greater or equal to 4 million cells/ml, more preferably to a cell density greater or equal to 6, 8, 10, 12, 15, 20, 25, 30, 35, or even 40 million cells/ml;

b)—performing a fed-batch culture by feeding the culture with a concentrated cell culture medium and/or at least one source of carbohydrate, preferably a sugar and/or glutamine, until the culture volume reaches the maximum vessel volume; and then

c) optionally, adding for the remaining days of animal cell culture in said vessel, marginal volume of at least one compound selected among one source of carbohydrate, preferably a sugar as an energy source and a solution of one or more amino-acids, preferably glutamine.

and wherein said animal cells are genetically modified to express said recombinant protein.

According to a preferred embodiment, the present invention relates to a process of production of recombinant protein, such as monoclonal antibody in a fed-batch culture in animal cells, preferably duck cell lines, and more preferably duck EB66 cell line, comprising the steps of:

a)—growing the cells, preferably in exponential phase, in a cell growth medium, optionally supplemented with defined supplements, in a culture volume corresponding to about 50% or less of the maximum vessel volume, to a cell density greater or equal to 10 million cells/ml, preferably 12 million cells/ml, more preferably 15 million cells/ml, or even more preferably to a cell density greater or equal to 20 million cells/ml;

b)—performing a fed-batch culture by feeding the culture with a concentrated cell culture medium, D-glucose and glutamine, until the culture volume reaches the maximum vessel volume; and then

c) optionally, adding for the remaining days of animal cell culture in said vessel, marginal volume of D-glucose and glutamine.

and wherein said animal cells are genetically modified to express said recombinant protein.

Generation of genetically modified cells to express recombinant proteins is well-known by the man skilled in the art. Methods which are well known to and practiced by those skilled in the art can be used to construct expression vectors containing sequences encoding the proteins and polypeptides of interest, as well as the appropriate transcriptional and translational control elements. These methods include in vitro recombinant DNA techniques, synthetic techniques, and in vivo genetic recombination. Such techniques are described for example in Sambrook et al. (1989, Molecular Cloning, A Laboratory Manual, Cold Spring Harbor Press, Plainview, N.Y.) and in Ausubel et al. (1989, Current Protocols in Molecular Biology, John Wiley & Sons, New York, N.Y.).

According to a preferred embodiment, when producing recombinant proteins, the cells of step a) are grown to a cell density greater or equal to 4 million cells/ml, greater or equal to 8 million cells/m, more preferably greater or equal to 10 million cell/ml, and even more preferably greater or equal to 20 million cell/ml.

The production of recombinant proteins, such as monoclonal antibody, in a fed-batch culture in animal cells according to the invention is performed at a temperature comprises between 32° C. to 39° C. depending of cell type. According to a preferred embodiment, step a) is performed at a temperature comprise between 35 and 39° C., more preferably approximately 37° C.; step b) and c) are performed at a lower temperature comprises below 37° C., preferably at 35° C., more preferably at 34° C., more preferably at 33° C., and even more preferably below or at 32° C.

The present method optionally includes harvesting the cultured cells during or subsequent to the period of continuous culture. Harvesting methods include, but are not limited to, centrifugation, settling, filtration and acoustic separation.

The invention also relates to the recombinant proteins produced by the process of the invention, and their uses as human and animal therapeutic and prophylactic drugs.

In preferred embodiment, the process of production of recombinant protein in a fed-batch culture in animal cells, is harvested at a time between 8 and 20 days (from the vessel seeding). It is expected that after 8 days already some increase in yield will be obtained as compared to the batch process, whereas processes longer than 20 days would likely suffer from a decrease in quality of the product obtained. The duration of the fed-batch process of recombinant protein, specially monoclonal antibody, is preferably less than 15 days post vessel seeding.

Examples of proteins of interest that can be advantageously produced by the method of this invention include, without limitation, cytokines, cytokine receptors, growth factors (e.g. EGF, HER-2, FGF-alpha, FGF-beta, TGF-alpha, TGF-beta, PDGF, IGF-1, IGF-2, NGF), growth factor receptors, including fragment of the protein thereof. Other non-limiting examples include growth hormones (e.g. human growth hormone, bovine growth hormone); insulin (e.g., insulin A chain and insulin B chain), pro-insulin, erythropoietin (EPO), colony stimulating factors (e.g. G-CSF, GM-CSF, M-CSF); interleukins (e.g. IL-1 through IL-12); vascular endothelial growth factor (VEGF) and its receptor (VEGF-R), interferons (e.g. IFN-alpha, beta and gamma), tumor necrosis factor (TNF) and their receptors (TNFR-1 and TNFR-2), thrombopoietin (TPO), thrombin, brain natriuretic peptide (BNP); clotting factors (e.g. FactorVIII, Factor IX, von Willebrands factor and the like), anti-clotting factors; tissue plasminogen activator (TPA), urokinase, follicle stimulating hormone (FSH), luteinizing hormone (LH), calcitonin, CD proteins (e. g., CD2, CD3, CD4, CD5, CD7, CD8, CD11a, CD11b, CD18, CD19, CD20, CD25, CD33, CD44, CD45, CD71, etc.), CTLA proteins (e.g. CTLA4); T-cell and B-cell receptor proteins, bone morphogenic proteins (BNPs, e.g. BMP-1, BMP-2, BMP-3, etc.), neurotrophic factors, e.g. bone derived neurotrophic factor (BDNF), neurotrophins, e.g. rennin, rheumatoid factor, RANTES, albumin, relaxin, macrophage inhibitory protein (e.g. MIP-1, MIP-2), viral proteins or antigens, surface membrane proteins, ion channel proteins, enzymes, regulatory proteins, antibodies, immunomodulatory proteins, (e.g. HLA, MHC, the B7 family), homing receptors, transport proteins, superoxide dismutase (SOD), G-protein coupled receptor proteins (GPCRs), neuromodulatory proteins, Alzheimer's Disease associated proteins and peptides, (e.g. A-beta) and others as known in the art. Fusion proteins and polypeptides, chimeric proteins and polypeptides, as well as fragments or portions, or mutants, variants, or analogs of any of the aforementioned proteins and polypeptides are also included among the suitable proteins, polypeptides and peptides that can be produced by the methods of the present invention.

In a preferred embodiment, the protein of interest is a glycoprotein, and preferably a viral protein. Example of viral proteins (subunits) that can be produced in the methods according to the invention include, without limitation, proteins from enterovirus, such as rhinovirus, aphtovirus, or poliomyelitis virus, herpes virus, such as herpes simplex virus, pseudorabies virus or bovine herpes virus, orthomyxovirus such as influenza virus, a paramyxovirus, such as newcastle disease virus, respiratory syncitial virus, mumps virus or a measles virus, retrovirus, such as human immunodeficiency virus or a parvovirus or a papovavirus, rotavirus or a coronavirus, such as transmissable gastroenteritisvirus or a flavivirus, such as tick-borne encephalitis virus or yellow fever virus, a togavirus, such as rubella virus or eastern-, western-, or venezuelean equine encephalomyelitis virus, a hepatitis causing virus, such as hepatitis A or hepatitis B virus, a pestivirus, such as hog cholera virus or a rhabdovirus, such as rabies virus. According to another embodiment, the protein of interest is a bacterial protein.

In another preferred embodiment, the biological product of interest is an antibody. The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and broadly encompasses naturally-occurring forms of antibodies (for example, IgD, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprises any modified or derivatised variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)₂ fragments, disulfide-linked Fvs (sdFv) fragments, anti-idiotypic (anti-Id) antibodies, intra-bodies, synthetic antibodies, and epitope-binding fragments of any of the above. The term “antibody” also refers to fusion protein that includes a region equivalent to the Fc region of an immunoglobulin.

Preferred antibodies within the scope of the present invention include those comprising the amino acid sequences of the following antibodies: anti-HER2 antibodies including antibodies comprising the heavy and light chain variable regions of huMAb 4D5-8 (Carteret al., Proc. Natl. Acad. Sci. USA, 89: 4285-4289 (1992), U.S. Pat. No. 5,725,856) or Trastuzumab such as HERCEPTIN™; anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108 or Tositumomab (BEXXAR); anti-IL-8 (St John et al., Chest, 103: 932 (1993), and International Publication No. WO 95/23865); anti-VEGF antibodies including humanized and/or affinity matured anti-VEGF antibodies such as the humanized anti-VEGF antibody huA4.6.1 AVASTIN™ (Kim et al., Growth Factors, 7: 53-64 (1992), International Publication No. WO 96/30046, and WO 98/45331); anti-PSCA antibodies (WO01/40309); anti-CD40 antibodies, including S2C6 and humanized variants thereof (WO00/75348); anti-CD11a (U.S. Pat. No. 5,622,700, WO 98/23761); anti-EGFR (chimerized or humanized 225 antibody as in WO 96/40210); anti-CD3 antibodies such as OKT3 (U.S. Pat. No. 4,515,893); anti-CD25 or anti-tac antibodies such as CHI-621 (SIMULECT) and (ZENAPAX) (See U.S. Pat. No. 5,693,762); anti-CD4 antibodies such as the cM-7412 antibody (Choy et al. Arthritis Rheum 39(1) : 52-56 (1996)); anti-CD52 antibodies such as CAMPATH-1H (Riechmann et al. Nature 332: 323-337 (1988); anti-carcinoembryonic antigen (CEA) antibodies such as hMN-14 (Sharkey et al. Cancer Res. 55 (23Suppl): 5935s-5945s (1995); anti-EpCAM antibodies such as 17-1A (PANOREX); anti-GpIIb/IIIa antibodies such as abciximab or c7E3 Fab (REOPRO); anti-RSV antibodies such as MEDI-493 (SYNAGIS); anti-CMV antibodies such as PROTOVIR; anti-hepatitis antibodies such as the anti-HepB antibody OSTAVIR; anti-human renal cell carcinoma antibody such as ch-G250; anti-human17-1A antibody (3622W94); anti-human colorectal tumor antibody(A33); anti-human melanoma antibody R24 directed against GD3 ganglioside; anti-human squamous-cell carcinoma (SF-25); and anti-human leukocyte antigen (HLA) antibodies such as SmartID10 and the anti-HLA DR antibody Oncolym(Lym-1).

According to a preferred embodiment, the transfected cells of the invention, and more specifically duck cell lines, and more specifically EB66 cells, are able to produce at least 5 pg/cell/day of immunoglobulin in batch culture, preferably at least 10 pg/cell/day of immunoglobulin in batch culture, more preferably at least 20 pg/cell/day of immunoglobulin in batch culture, even more preferably at least 30 pg/cell/day of immunoglobulin in batch culture. The two chains assemble within the cell and are then secreted into the culture medium as functional antibody.

Interestingly, the inventors have demonstrated that the antibody, the antibody fragment, or the fusion proteins that include a region equivalent to a Fc region of an immunoglobulin, produced in a duck cell line, such as EB66 cells, by the method of the invention have increased Fc-mediated cellular toxicity. For example, antibody of IgG1 subtype, produced in EB66 cells, have an increased ADCC activity compared to the same antibody produced in hybridoma (e.g NS0) and CHO cells. This is achieved by providing the antibodies of interest with the duck glycosylation pattern.

In particular, the transfected duck cells, more specifically EB66 cells of the invention, allow to express a large proportion of antibodies or fragment thereof, carrying a common N-linked oligosaccharide structure of a biantennary-type that comprises long chains with terminal GlcNac that are highly galactosylated and non-fucosylated and which confer strong ADCC activity to antibodies. Among a recombinant antibody population produced in duck cells, more specifically in EB66 cells, the proportion of non-fucosylated antibodies represent at least 30%, more preferably at least 45%, more preferably at least 55%, and more preferably at least 65% of the antibodies or higher. More precisely, the invention provides a recombinant monoclonal antibody produced by duck cell line, preferably duck EB66 cell line, wherein said antibody is characterized as having approximately 45% or more of non-fucosylated N-linked oligosaccharides structures in the antibody population produced in duck cells.

The instant invention relates to the biological product of interest according the invention as a medicament. The invention also covers the use of a biological product (i.e viral vaccine, recombinant protein, monoclonal antibody . . . ) obtained by the process of the invention, for the preparation of a pharmaceutical composition for the prevention or the treatment of human and animal diseases. Such pharmaceutical compositions preferably include, in addition to the biological product, a physiologically acceptable diluent or carrier.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims. For the remainder of the description, reference will be made to the legend to the figures below.

### FIGURES

FIG. 1: GLUCOSE FEDBATCH PROCESS USING CONCENTRATED MEDIUM FOR MONOCLONAL ANTIBODY PRODUCTION IN EB66 CELL LINE

FIG. 1A: in a simple batch process, monoclonal antibody producing EB66 cells have a short viability (approx. 5 days) which decreases after day 5. EB66 cells reach a maximum viable cell density of approximately 8 million cell/ml. The yield of Ig1 expressed in EB66 cells reaches 18 ug/ml.

FIG. 1B: in a glucose fedbatch process using concentrated medium, monoclonal antibody producing EB66 cells have a longer cell viability (approx. 8 days). EB66 cells reach a maximum viable cell density of approximately 40 million cell/ml. The yield of Ig1 expressed in EB66 cells reaches approximately 1 g/l. The fedbatch process of the invention allows a 90 fold increase in monoclonal antibody production yield compared to a regular batch process.

FIG. 2: METABOLIC ANALYSIS OF GLUCOSE FEDBATCH PROCESS USING CONCENTRATED MEDIUM FOR MONOCLONAL ANTIBODIES PRODUCTION IN EB66 CELL LINE

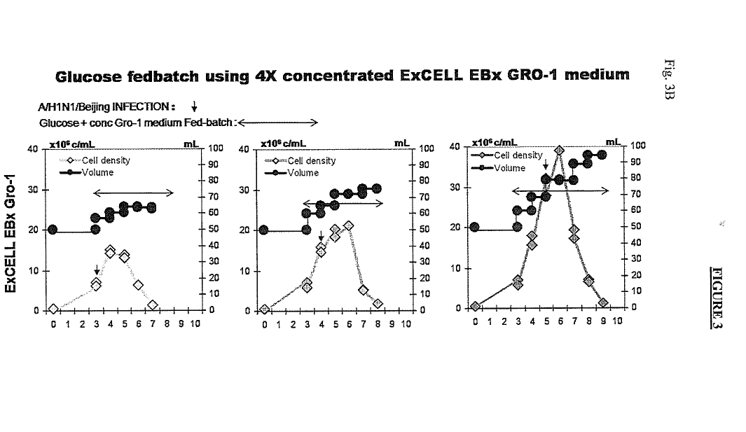

The metabolic profile of EB66 cells culture expressing IgG1 monoclonal antibodies shows remarkable features: No accumulation of lactate or ammonium even at a high viable cell density (>40 million cell/ml) and limited consumption of glutamine.

FIG. 3: PRODUCTION OF A/H1N1/BEIJING INFLUENZA VIRUS STRAIN IN EB66 CELL LINE USING BATCH PROCESS vs GLUCOSE FEDBATCH PROCESS

FIG. 3A: in the former standard process, EB66 cells were grown in ExCELL EBx GRO-1 cell growth medium (SAFC Biosciences) during 3 days up to a viable cell density of approximately 6 million cells/ml, then infected with influenza virus strain A/H1N1/Beijing/262/95. One hour after virus infection, 2 volumes of fresh virus production medium (SAFC Biosciences) was added to the Erlenmeyer flasks. A day 3 post-infection, the maximal viable cell density is approximately 9 million cells/ml.

FIG. 3B: EB66 cells (passage p142) were seeded in Shaken Erlenmeyers (250 ml) in ExCELL EBx GRO-I medium supplemented with 2.5 mM glutamine at day 0 at a cell concentration of 0.5×10exp6 cells/ml. EB66 cells were grown as a batch culture in a fixed volume until the day 3, 4 or 5, when the infection with A/H1N1/Beijing/262/95 influenza virus (multiplicity of infection 10⁻⁴ TCID50/cell) is performed. A glucose fedbatch at 8 g/l and a daily addition of ExCELL EBx GRO-I medium 4× concentrated medium is initiated at day 3, in order to reach maximum vessel volume at day 7 post infection. When the infection is performed at day 3, 4 and 5, maximum viable cell density is respectively of approximately 15 million cells/ml, 20 million cells/ml and 40 million cells/ml.

FIG. 4: PRODUCTION OF A/H1N1/BEIJING HAEMAGLUTININ IN STANDARD PROCESS VS GLUCOSE FEDBATCH PROCESS

Haemaglutinin titer measured by serial radial immuno-diffusion assay and expressed in ug/ml of cell culture supernatant obtained at different days post-infection. FIG. 4A: standard process; FIG. 4B: glucose fed-batch using concentrating medium.

FIG. 5: PRODUCTION OF A/H3N2/WISCONSIN INFLUENZA VIRUS STRAIN IN EB66 CELL LINE USING BATCH PROCESS vs GLUCOSE FEDBATCH PROCESS IN ERLENMEYER

Left panel: in the former standard process, EB66 cells were grown in ExCELL EBx GRO-1 cell growth medium (SAFC Biosciences) during 3 days up to a viable cell density of approximately 6 million cells/ml, then infected with influenza virus strain A/H3N2/Wisconsin. One hour after virus infection, 2 volumes of fresh virus production medium (SAFC Biosciences) was added to the Erlenmeyer flasks. A day 3 post-infection, the maximal viable cell density is approximately 9 million cells/ml.

Right Panel: EB66 cells (passage p142) were seeded in Shaken Erlenmeyers (250 ml) in ExCELL EBx GRO-I medium supplemented with 2.5 mM glutamine at day 0 at a cell concentration of 0.5×10exp6 cells/ml. EB66 cells were grown as a batch culture in a fixed volume until the day 3 when a glucose fedbatch at 10 or at 15 g/l, a glutamine fedbatch at 2.5 mM and a daily addition of ExCELL EBx GRO-I medium 4× concentrated medium is initiated. The infection with A/H3N2/Wisconsin influenza virus (multiplicity of infection 10⁻⁴ TCID50/cell) is performed at day 5, in order to reach maximum vessel volume at day 7 post seeding. Maximum viable cell density obtained with a glucose fedbatch at 10 g/l and 15 g/l is approximately 20 million cells/ml.

FIG. 6: PRODUCTION OF A/H3N2/WISCONSIN HAEMAGLUTININ IN STANDARD PROCESS VS GLUCOSE FEDBATCH PROCESS

Haemaglutinin titer measured by serial radial immuno-diffusion assay and expressed in ug/ml of cell culture supernatant obtained at different days post-infection. The absolute haemagglutinin (HA) productivity value obtained in 250 ml Erlenmeyers with the glucose fedbatch process (10 g/l and 15 g/l) using concentrated medium is much higher (approx. 80 ug/ml of HA) than the one obtained with standard process (approx. 30 ug/ml of HA).

FIG. 7: PRODUCTION OF A/H3N2/WISCONSIN INFLUENZA VIRUS STRAIN IN EB66 CELL LINE USING BATCH PROCESS vs GLUCOSE FEDBATCH PROCESS IN 2 L-BIOREACTOR

FIG. 7A: in the former standard process, EB66 cells were grown in ExCELL EBx GRO-1 cell growth medium (SAFC Biosciences) during 3 days up to a viable cell density of approximately 6 million cells/ml, then infected with influenza virus strain A/H3N2/Wisconsin. One hour after virus infection, 2 volumes of fresh virus production medium (SAFC Biosciences) was added to the Erlenmeyer flasks. A day 3 post-infection, the maximal viable cell density is approximately 9 million cells/ml.

FIG. 7B: EB66 cells (passage p142) were seeded in Shaken Erlenmeyers (250 ml) in ExCELL EBx GRO-I medium supplemented with 2.5 mM glutamine at day 0 at a cell concentration of 0.5×10exp6 cells/ml. EB66 cells were grown as a batch culture in a fixed volume until the day 3 when a glucose fedbatch at 8 g/l, a glutamine fedbatch at 2.5 mM and a daily addition of ExCELL EBx GRO-I medium 4× concentrated medium is initiated. The infection with A/H3N2/Wisconsin influenza virus (multiplicity of infection 10⁻⁴ TCID50/cell) is performed at day 4, in order to reach maximum vessel volume at day 6 post seeding. Maximum viable cell density obtained with a glucose fedbatch at 8/l is approximately 25-30 million cells/ml.

FIG. 8: PRODUCTION OF A/H3N2/WISCONSIN HAEMAGLUTININ IN STANDARD PROCESS VS GLUCOSE FEDBATCH PROCESS IN 2 L BIOREACTOR

Haemaglutinin titer measured by serial radial immuno-diffusion assay and expressed in ug/ml of cell culture supernatant obtained at different days post-infection. The absolute haemagglutinin (HA) productivity value obtained in 2 L Bioreactor with the glucose fedbatch process (8 g/l) using concentrated medium is much higher (approx. 100 ug/ml of HA) than the one obtained with standard process (approx. 30 ug/ml of HA).

### EXAMPLES

Duck EB66 cell line, which has been established according to the process described in patent applications WO2008/129058 & WO2008/142124, were used in the following examples.

### Example 1

### Glucose Fedbatch Process using Concentrated Medium for Monoclonal Antibody Production

In the fed-batch process of the invention, EB66 cells were seeded in 2 L-stirred tank bioreactor (Applikon™) at a cell concentration of 0.5×10exp6 cells/ml in a cell growth medium (Hyclone) and allowed to grow as a batch culture in a fixed volume. When the viable cell density is greater than 4 millions cells/ml, usually at day 3, a glucose fed-batch process is implemented until for example day 8. The fed-hatch process comprises:
daily addition of 4× concentrated ExCELL EBx GRO-1 cell culture medium (SAFC Biosciences Cat. No 14530c). The formulation of 4× concentrated ExCell EBx GRO-I medium was obtained by a 4 fold increase of the concentration of each component of ExCELL EBx GRO-1 medium, except the components which are involved in, or act on, the osmotic pressure and/or the osmolality, such as salts and buffering agents, which are 1× concentrated in the 4× concentrated ExCell EBx GRO-I medium.
daily addition of glucose to maintain glucose concentration in the medium at 10 g/l concentration;
daily addition of glutamine to maintain glutamine concentration in the medium at 2.5 mM concentration.

A standard batch process was run in parallel which consists of seeding EB66 cells in 2 L-stirred tank bioreactor (Applikon™) at a cell concentration of 0.5×10exp6 cells/ml in a cell growth medium (Hyclone), then growing cells without nutrient feed in a fixed volume during 8 days.

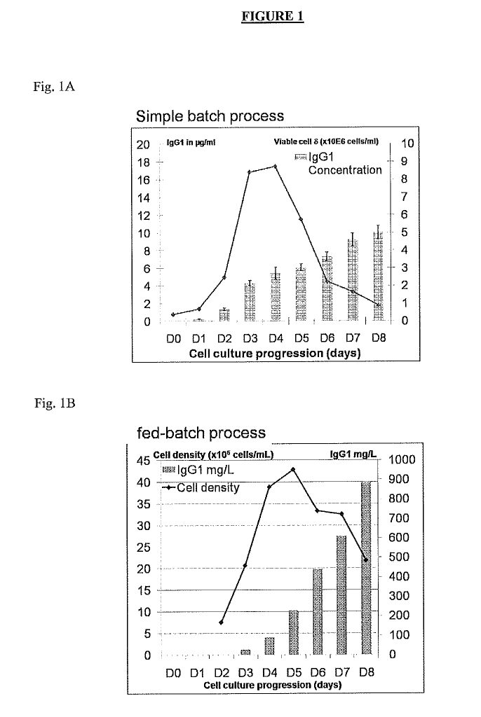

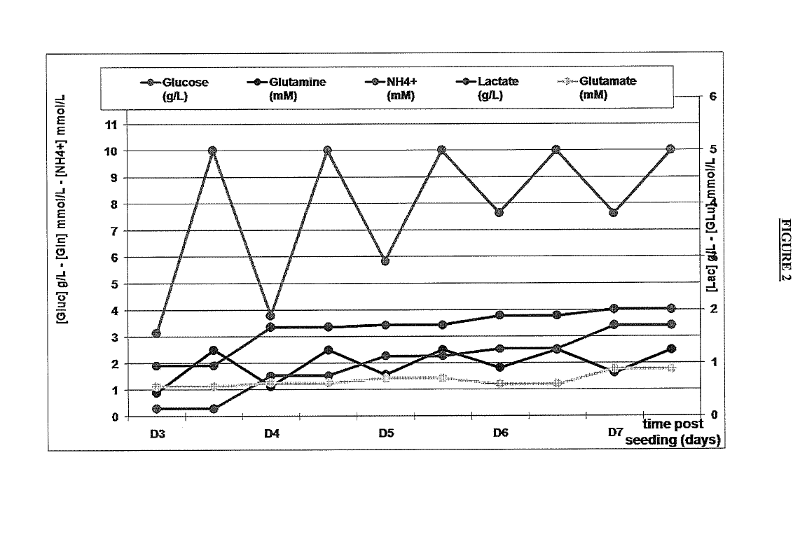

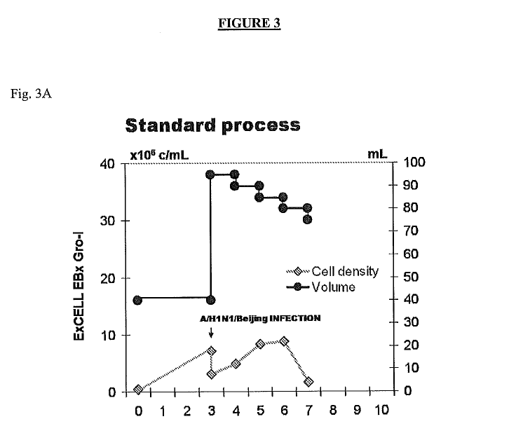

The standard batch process of IgG1 monoclonal antibody in EB66 cells (FIG. 1) allows to reach a short cell viability (approx. 5 days), a cell density of approximately 8 million cells/ml and low monoclonal antibody production yield of approximately 18 ug/ml.

In a fedbatch process of the invention, monoclonal antibody producing EB66 cells have a longer cell viability (approx. 8 days). EB66 cells reach a maximum viable cell density of approximately 40 million cell/ml. The yield of Ig1 expressed in EB66 cells reaches approximately 1 g/l.

The fedbatch process of the invention allows a 90 fold increase in monoclonal antibody production yield compared to a regular batch process.

Very interestingly, no accumulation of lactate or ammonium (even at a very high viable cell density of >40 million cell/ml) is observed with monoclonal antibody producing EB66 cells culture. This appears to be a characteristic of duck cell line such as EB66 cells. In addition, duck cell lines, such as EB66 cells, have also limited consumption of glutamine.

The inventors thus demonstrated that very high cell densities of duck cell, such as EB66 cells, can be achieved by a better control of nutrient consumptions. Today experimental data allowed to reach cell densities up to 70 million cells/ml without accumulation of lactate or ammonium in the culture. Therefore, duck cell line, such as EB66, have the remarkable capability to grow in conditions of high glucose concentrations (>8 g/l) and to accumulate no or low lactate and ammonium, allowing to reach high cell densities.

### Example 2

### Glucose Fedbatch Process using Concentrated Medium for A/H1N1/Beijing/262/95 Influenza Virus Strain Production

2.1—Standard Process of Production of A/H1N1/Beijing/262/95 Influenza Virus Strain

The Fedbatch process of the invention was run in parallel to the former standard process that includes the following steps: proliferating EB66 cell line in cell growth medium, infecting the cells with influenza virus, then adding virus production medium and further culturing infected cells to allow virus replication.

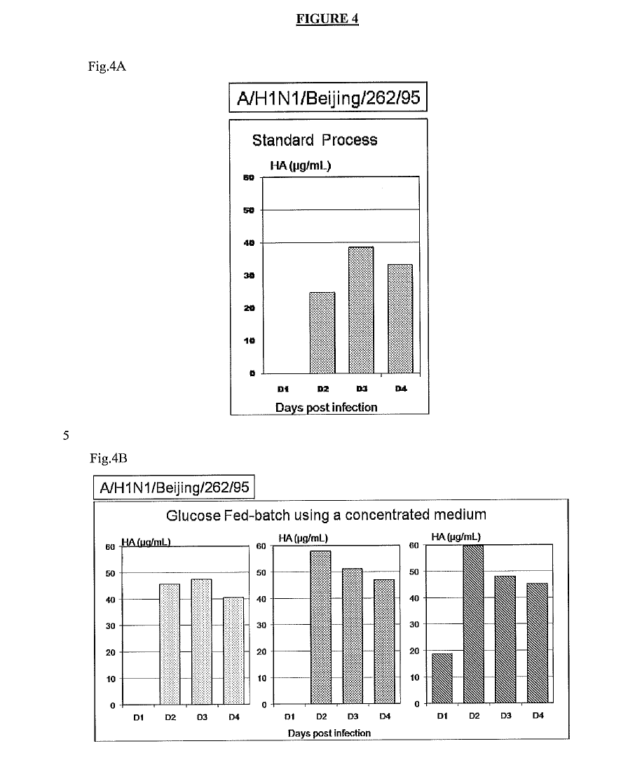

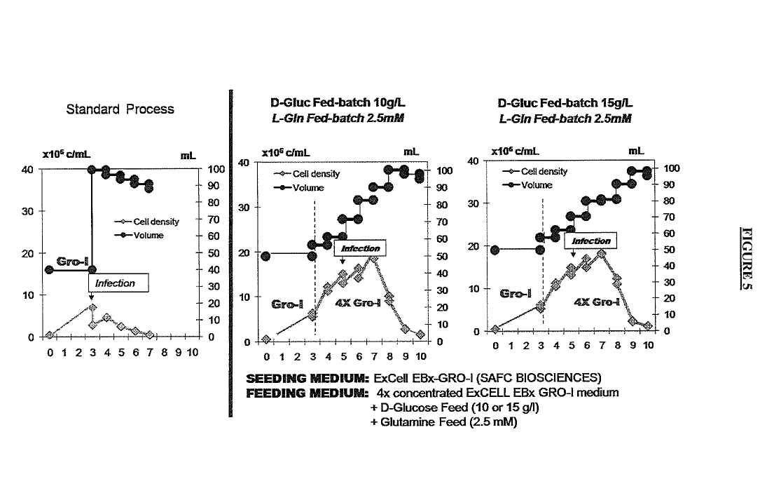

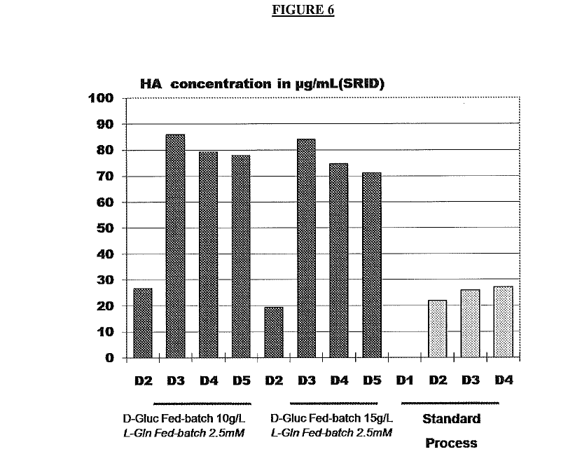

Briefly, duck EB66 cells (passage 142) were grown in Shaken Erlenmeyers (250 ml) in one volume of (1×) SAFC BIOSCIENCES ExCELL EBx GRO-I animal serum free medium (Cat No 14530c) supplemented with 2.5 mM glutamine. At day 3 post seeding, when the cell density is around or greater than 6 million cells/ml, influenza virus infection is performed at a multiplicity of infection of 10⁻⁴ TCID50/ml. One hour after virus infection, two volumes of fresh animal serum free virus production medium were added (FIG. 3A). The maximum viable cell density obtained is approximately 9 million cells/ml.

2.2—Glucose Fedbatch Process using Concentrated Medium for the Production of A/H1N1/Beijing/262/95 Influenza Virus Strain

EB66 cells (passage p142) were seeded in Shaken Erlenmeyers (250 ml) in ExCELL EBx GRO-I medium supplemented with 2.5 mM glutamine at day 0 at a cell concentration of 0.5×10exp6 cells/ml. EB66 cells were grown as a batch culture in a fixed volume until the day 3, 4 or 5, when the infection with A/H1N1/Beijing/262/95 influenza virus (multiplicity of infection 10⁻⁴ TCID50/cell) is performed.

A glucose fedbatch at 8 g/l, a glutamine fedbatch at 2.5 mM and a daily addition of ExCELL EBx GRO-I medium 4× concentrated medium are initiated at day 3, in order to reach maximum vessel volume at day 7 post infection.

The formulation of 4× concentrated ExCell EBx GRO-I medium was obtained by a 4 fold increase of the concentration of each component of ExCELL EBx GRO-1 medium, except the components which are involved in, or act on, the osmotic pressure and/or the osmolality, such as salts and buffering agents, which are 1× concentrated in the 4× concentrated ExCell EBx GRO-I medium.

When the infection is performed at day 3, 4 and 5, maximum viable cell density is respectively of approximately 15 million cells/ml, 20 million cells/ml and 40 million cells/ml (FIG. 3B).

2.3—Production of A/H1N1/Beijing/262/95 Haemaggutinin

The concentration of influenza haemagglutinin in the cell culture medium was determined by the SRID method as described by Wood et al. (An improved single-radial-immunodiffusion technique for the assay of influenza haemagglutinin antigen: application for potency determinations of inactivated whole virus and subunit vaccines” J. Biol. Stand. 1977, 5(3):237-247).

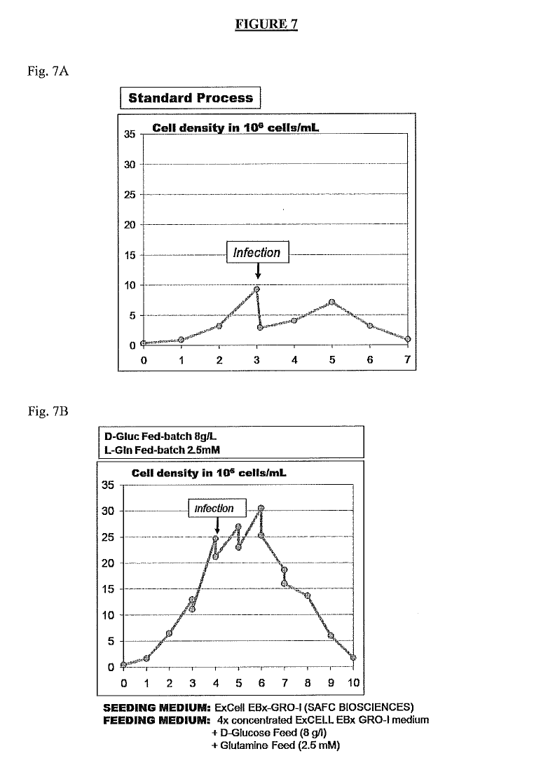

FIG. 4 presents the SRID analysis performed on samples collected at day 1, day 2, day 3 and day 4 post-infection and obtained with the former process of influenza virus production in EB66 cells (FIG. 4A) and the glucose fedbatch process using concentrated medium (FIG. 4B). The absolute haemagglutinin (HA) productivity value is enhanced with the elevation of the EB66 biomass at time of infection.

### Example 3

### Glucose Fedbatch Process using Concentrated Medium for a A/H3N2/Wisconsin Influenza Virus Strain Production in Skahen Erlenmeyer

3.1—Former Standard Process of Production of A/H3N2/Wisconsin Influenza Virus Strain

The standard process described in Example 2.1 was run with this second A/H3N2/Wisconsin influenza virus strain. The maximum viable cell density obtained is approximately 8 million cells/ml.

3.2—Glucose Fedbatch Process using Concentrated Medium for the Production of A/H3N2/Wisconsin Influenza Virus Strain

EB66 cells (passage p142) were seeded in shaken Erlenmeyers (250 ml) in ExCELL EBx GRO-I medium supplemented with 2.5 mM glutamine at day 0 at a cell concentration of 0.5×10exp6 cells/ml. EB66 cells were grown as a batch culture in a fixed volume until the day 3 when the glucose fedbatch was initiated.

The infection with A/H3N2/Wisconsin influenza virus (multiplicity of infection 10⁻⁴ TCID50/cell) was performed at day 5 post seeding when the EB66 cells almost reached maximum viable cell density (approx. 15 million cells/ml).

A glucose fedbatch either at 10 g/l or 15 g/l, a glutamine fedbatch at 2.5 mM and a daily addition of ExCELL EBx GRO-I medium 4× concentrated medium are initiated at day 3, in order to reach maximum vessel volume at day 7 post infection.

The formulation of 4× concentrated ExCell EBx GRO-I medium was obtained by a 4 fold increase of the concentration of each component of ExCELL EBx GRO-1 medium, except the components which are involved in, or act on, the osmotic pressure and/or the osmolality, such as salts and buffering agents, which are 1× concentrated in the 4× concentrated ExCell EBx GRO-I medium.

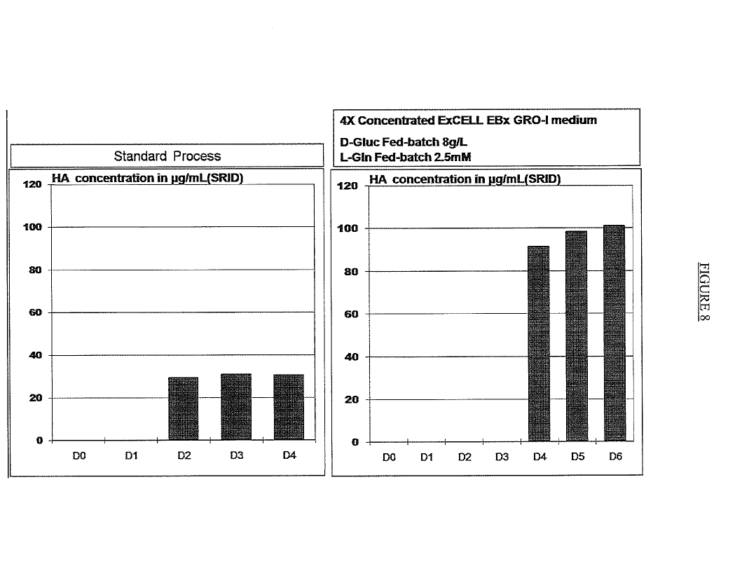

No major difference in the maximum viable cell density (i.e 20 million cells/ml at day 4 post-infection) is observed when performing a glucose fedbatch at 10 or 15 g/l (FIG. 5).

3.3—Production of A/H3N2/Wisconsin Haemaggutinin

The concentration of influenza haemagglutinin in the cell culture medium was determined by the SRID method as described by Wood et al.

FIG. 6 presents the SRID analysis performed on samples collected at day 1, day 2, day 3, day 4 and day 5 post-infection and obtained either with the standard process of influenza virus production in EB66 cells or the glucose fedbatch process (10 g/l and 15 g/l) using concentrated medium.

The absolute haemagglutinin (HA) productivity value obtained in 250 ml Erlenmeyers with the glucose fedbatch process (10 g/l and 15 g/l) using concentrated medium is much higher (approx. 80 ug/ml of HA) than the one obtained with standard process (approx. 30 ug/ml of HA).

The glucose fedbatch process using concentrated medium allows to get robust cell culture of EB66 cells that reach high viable cell density (i.e 20 million cells/ml at day 4 post-infection). The increase of biomass at time of infection with the glucose fedbatch process using concentrated medium, allows to get higher haemagglutinin (HA) productivity compared to the standard process.

### Example 4

### Glucose Fedbatch Process using Concentrated Medium for A/H3N2/Wisconsin Influenza Virus Strain Production in 2 L-Stirred Tank Bioreactor

4.1—Former Standard Process of Production of A/H3N2/Wisconsin Influenza Virus Strain

The standard process described in Example 2.1 was run with this second A/H3N2/Wisconsin influenza virus strain. The maximum viable cell density obtained is approximately 10 million cells/ml (FIG. 7A).

4.2—Glucose Fedbatch Process using Concentrated Medium for the Production of A/H3N2/Wisconsin Influenza Virus Strain

EB66 cells (passage p142) were seeded in 2 L-Bioreactor in ExCELL EBx GRO-I medium supplemented with 2.5 mM glutamine at day 0 at a cell concentration of 0.5×10exp6 cells/ml. EB66 cells were grown as a batch culture in a fixed volume until the day 3 when the glucose fedbatch was initiated.

The infection with A/H3N2/Wisconsin influenza virus (multiplicity of infection 10⁻⁴ TCID50/cell) was performed at day 4 post seeding when the EB66 cells almost reached maximum viable cell density (approx. 22 million cells/ml).

A glucose fedbatch either at 8 g/l, a glutamine fedbatch at 2.5 mM and a daily addition of ExCELL EBx GRO-I medium 4× concentrated medium are initiated at day 3, in order to reach maximum vessel volume at day 6 post infection (FIG. 7B).

4.3—Production of A/H3N2/Wisconsin Haemaggutinin

The concentration of influenza haemagglutinin in the cell culture medium was determined by the SRID method as described by Wood et al.

FIG. 8 presents the SRID analysis performed on samples collected at day 1, day 2, day 3, day 4, day 5 and day 6 post-infection and obtained either with the standard process of influenza virus production in EB66 cells or the glucose fedbatch process (8 g/l) using concentrated medium.

The absolute haemagglutinin (HA) productivity value obtained in 250 ml Erlenmeyers with the glucose fedbatch process (8 g/l) using concentrated medium is much higher (approx. 100 ug/ml of HA) than the one obtained with standard process (approx. 30 ug/ml of HA).

The glucose fedbatch process using concentrated medium allows to get robust cell culture of EB66 cells that reach high viable cell density (i.e 25 million cells/ml at day 4 post-infection). The increase of biomass at time of infection with the glucose fedbatch process using concentrated medium, allows to get higher haemagglutinin (HA) productivity compared to the standard process.

1. A process for the fed-batch culture of animal cells comprising the steps of:
a) growing the cells in a cell growth medium, optionally supplemented with defined supplements, in a culture volume corresponding to a maximum of 75% of the maximum vessel volume, to a cell density greater or equal to 4 million cells/ml; and wherein said process comprises the steps of:
b) performing a fed-batch culture by feeding the culture with a concentrated cell culture medium and/or at least one source of carbohydrate and/or glutamine, until the culture volume reaches the maximum vessel volume; and then
c) optionally, adding marginal volume of at least one compound selected among one source of carbohydrate as an energy source and a solution of one or more amino-acids.

2. The process according to claim 1, wherein said animal cells are duck cell line cells.

3. The process according to claim 1 or claim 2, wherein the cell density at step a) is greater or equal to 10 million cells/ml.

4. The process according to claim 1, wherein the sugar is glucose and wherein the glucose concentration in the cell growth medium is maintained by fedbatch at a final concentration of at least 8 g/l.

5. The process according to claim 1, wherein the glutamine concentration in the medium is maintained by fed-batch at a final concentration of about 2.5 mM.

6. The process according to claim 1, wherein the cell density at the end of step b) is above or equal to 25 million cells/ml.

7. The process according to claim 1, wherein the maximum vessel volume is reached at day 6 post vessel seeding.

8. The process according to claim 1, wherein said process lasts less than 15 days.

9. The process according to claim 1, for the production of a recombinant protein, wherein said animal cells are genetically modified to express said recombinant protein, and wherein said process comprises the additional and final step of harvesting said recombinant protein from the nutrient medium.

10. The process according to claim 9, wherein said recombinant protein is a monoclonal antibody.

11. The process according to claim 1 for the production of virus in said animal cells, wherein said process comprises the additional step of infecting the culture of animal cells with said virus, and wherein the infection step is carried out either during step a), between step a) and b), or during step b).

12. The process according to claim 11, wherein the infection step is carried out during step b).

13. The process according to claim 11, wherein the virus is selected from naturally occurring viruses, attenuated viruses, reassortant viruses, recombinant viruses, and viral vectors.

14. The process according to claim 11, wherein the virus is selected from the group consisting of adenoviruses, hepadnaviruses, herpes viruses, orthomyxoviruses, papovaviruses, paramyxoviruses, picomaviruses, poxviruses, reoviruses, and retroviruses.

15. The process according to claim 11, wherein the concentrated culture medium added in step b) is a 4× concentrated culture medium, optionally supplemented with glucose and/or glutamine and step c) comprises the addition of glutamine feeds, to maintain a concentration of 2.5 mM of glutamine and the addition of glucose feeds to maintain a concentration above 8 g/l.

16. The process according to claim 1 or claim 2, wherein the cell density at step a) is greater or equal to 15 million cell/ml.

17. The process according to claim 1 or claim 2, wherein the cell density at step a) is greater or equal to 20 million cell/ml.

18. The process according to claim 11, wherein the concentrated culture medium added in step b) is a 4× concentrated culture medium, optionally supplemented with glucose and/or glutamine and step c) comprises the addition of glutamine feeds, to maintain a concentration of 2.5 mM of glutamine and the addition of glucose feeds to maintain a glucose concentration of 10 g/l.
